(19) United States
(12) Patent Application Publication
(10) Pub. No.: US 2024/0261570 A1
(43) Pub. Date: Aug. 8, 2024

(54) CLOSED-LOOP NEUROSTIMULATION SYSTEM AND METHOD

(71) Applicant: City University of Hong Kong, Hong Kong (HK)

(72) Inventors: Chi Him Eddie MA, Hong Kong (HK); Kumar GAJENDRA, Hong Kong (HK); Vivian Yen Yen CHU, Hong Kong (HK)

(21) Appl. No.: 18/163,288

(22) Filed: Feb. 2, 2023

(51) Int. Cl. A61N 1/36; A61N 1/05

(52) U.S. Cl. CPC A61N 1/36003; A61N 1/0534; A61N 1/36135; A61N 1/36171

(57) ABSTRACT

The present disclosure provides a closed-loop DBS system and method for regulating motor symptoms of a subject. The DBS system comprises: neural electrodes for being implanted in deep brain nuclei of the subject and a closed-loop stimulation generator in communication with the neural electrodes. The stimulation generator includes: a data acquisition unit configured to receive the LFP signals transmitted from the neural electrodes and convert the LFP signals to LFP data; a processing unit in communication configured to analyze the LFP data to determine presence of abnormal beta band oscillation and generate a DBS signal upon determining presence of abnormal beta band oscillation; and a pulse generation unit configured to generate, in response to the DBS signal, the DBS pulses to the deep brain nuclei through the neural electrodes. The provided system and method can save stimulation time and avoid adverse side effects of continuous stimulation while maintaining treatment efficacy.

### FIELD OF THE INVENTION

The present invention generally relates to neurostimulation. More specifically the present invention related to a closed-loop neurostimulation system and method for alleviating motor symptoms associated with neurodegenerative disorders.

### BACKGROUND OF THE INVENTION

Deep brain stimulation (DBS) is a medical treatment that uses electrical current to stimulate specific neurons in a brain to alleviate motor symptoms such as tremor, rigidity, stiffness, slowed movement, and walking problems associated with neurodegenerative disorders such as Parkinson's disease (PD) and epilepsy. Conventional open-loop DBS system delivers continuous stimulation regardless of changes in physiologic state, exhibits adverse side effects due to excessive current flow to adjacent structures, reduces the battery lifetime of stimulators and increases frequency of replacement surgery.

Accordingly, there is a strong need for a closed-loop DBS system which can reduce stimulation time and avoid adverse side effects of continuous stimulation while maintaining treatment efficacy.

### SUMMARY OF THE INVENTION

It is an objective of the present invention to provide a closed-loop DBS system and method which can reduce stimulation time and avoid adverse side effects of continuous stimulation while maintaining treatment efficacy for regulating motor symptoms of a subject.

In accordance with a first aspect of the present invention, the closed-loop DBS system comprises: one or more neural electrodes for being implanted in a group of deep brain nuclei of the subject to record local filed potential (LFP) signals generated in the deep brain nuclei and deliver deep brain stimulation (DBS) pulses to the deep brain nuclei; and a closed-loop stimulation generator in communication with the neural electrodes and including: a data acquisition unit in communication with the neural electrodes and configured to: receive the LFP signals transmitted from the neural electrodes; and convert the LFP signals to LFP data; a processing unit in communication with the data acquisition unit and configured to: receive the LFP data from the data acquisition unit; analyze the LFP data to determine presence of abnormal beta band oscillation; and generate a DBS signal upon determining presence of abnormal beta band oscillation; and a pulse generation unit in communication with the data processing unit and the neural electrodes and configured to: receive the DBS signal from the processing unit; and generate, in response to the DBS signal, the DBS pulses to the deep brain nuclei through the neural electrodes.

In accordance with one embodiment of the first aspect of the present invention, the processing unit is implemented with a field-programmable gate array.

In accordance with one embodiment of the first aspect of the present invention, the processing unit is further configured to: calculate an averaged LFP power based on the received LFP data; compare the averaged LFP power against a reference LFP power; and determine that the subject has abnormal beta band oscillation if the averaged beta band LPF power is higher than the reference LFP power.

In accordance with one embodiment of the first aspect of the present invention, the averaged LFP power is calculated from a beta band LFP power spectrum in a range from 13 to 40 Hz.

In accordance with one embodiment of the first aspect of the present invention, the averaged LFP power is calculated from a low beta band LFP power spectrum in a range from 13 to 20 Hz.

In accordance with one embodiment of the first aspect of the present invention, the averaged LFP power is calculated from a high beta band LFP power spectrum in a range from 20 to 40 Hz.

In accordance with one embodiment of the first aspect of the present invention, the neural electrodes communicate with the processing unit via a wireless network.

In accordance with one embodiment of the first aspect of the present invention, the neural electrodes communicate with the processing unit via a wired network.

In accordance with one embodiment of the first aspect of the present invention, the data acquisition unit is further configured to package the LFP data using user datagram protocol (UDP) and transmitted packaged LFP data the processing unit.

In accordance with one embodiment of the first aspect of the present invention, the data acquisition unit comprises a local memory for storing and queuing up the packaged LFP data.

In accordance with a second aspect of the present invention, the closed-loop neurostimulation method comprises: implanting one or more neural electrodes in a group of deep brain nuclei of the subject; sensing, by the neural electrodes, local filed potential (LFP) signals generated in the deep brain nuclei of the subject; acquiring and converting, by a data acquisition unit, the detected LFP signals to LFP data; packaging, by the data acquisition unit, the LFP data into data packets; queueing, by the data acquisition unit, the data packets in a memory; receiving, by a processing unit, the queued data packets from the memory; unpacking, by the processing unit, the data packets to retrieve the LFP data; analyzing, by the processing unit, the retrieved LFP data to determine presence of abnormal beta band oscillation; commanding, by the processing unit, a pulse generation unit to generate DBS pulses to the neural electrodes upon presence of abnormal beta band oscillation; and applying, through the neural electrodes, deep brain stimulation (DBS) pulses to the deep brain nuclei of the subject.

In accordance with one embodiment of the second aspect of the present invention, analyzing the retrieved LFP data to determine presence of abnormal beta band oscillation comprises: calculating an averaged LFP power based on the received LFP data; comparing the averaged LFP power against a reference LFP power; and determining that the subject has abnormal beta band oscillation if the averaged beta band LPF power is higher than the reference LFP power.

In accordance with one embodiment of the second aspect of the present invention, the averaged LFP power is calculated from a beta band LFP power spectrum in a range from 13 to 40 Hz.

In accordance with one embodiment of the second aspect of the present invention, the averaged LFP power is calculated from a low beta band LFP power spectrum in a range from 13 to 20 Hz.

In accordance with one embodiment of the second aspect of the present invention, the averaged LFP power is calculated from a high beta band LFP power spectrum in a range from 20 to 40 Hz.

In accordance with one embodiment of the second aspect of the present invention, the LFP data are packaged in real-time using a sliding window method.

In accordance with one embodiment of the second aspect of the present invention, the memory a local storage in the data acquisition unit.

In accordance with one embodiment of the second aspect of the present invention, the memory is a remote data-store.

Overall, the provided closed-loop DBS system and method can regulate motor symptoms to normal level in rotenone-induced as well as 6-OHDA-induced Parkinson's disease (PD) mouse models as evaluated by open filed, narrow beam, pole climb test. Compared with the open-loop DBS, the provided closed-loop DBS system and method requires 69.3% of total stimulation time in 6-OHDA-induced PD models and 55.4% of total stimulation time in rotenone-induced PD models.

### BRIEF DESCRIPTION OF THE DRAWINGS

Aspects of the present disclosure may be readily understood from the following detailed description with reference to the accompanying figures. The illustrations may not necessarily be drawn to scale. That is, the dimensions of the various features may be arbitrarily increased or reduced for clarity of discussion. There may be distinctions between the artistic renditions in the present disclosure and the actual apparatus due to manufacturing processes and tolerances. Common reference numerals may be used throughout the drawings and the detailed description to indicate the same or similar elements.

FIG. 1 depicts a block diagram of a closed-loop neurostimulation system according to one embodiment of the present invention.

FIG. 2 depicts a schematic diagram of a FPGA board with web function used for implementing a processing unit according to one embodiment of the present invention.

FIG. 3 depicts a schematic diagram of a closed-loop neurostimulation system according to one embodiment of the present invention.

FIG. 4 depicts how LFP data are packaged using user datagram protocol (UDP) for data transmission according to one embodiment of the present invention.

FIG. 5 depicts a flowchart of a method 500 according to one embodiment of the present invention.

FIG. 6 depicts how data packets are generated in real-time by using sliding window method according to one embodiment of the present invention.

FIG. 7 depicts an experiment paradigm for the evaluating effect of DBS on upstream (putamen) and downstream (STN/GPi) pathway of motor control in 6-OHDA induced PD mice models.

FIG. 8A depicts LFP powers of the control models, PD (no DBS) models and PD-Putamen DBS models, respectively.

FIG. 8B depicts spectrogram for the control models, PD (no DBS) models and PD-Putamen DBS models, respectively.

FIG. 8C depicts averaged LFP power of low beta band of the control models, PD (no DBS) models and PD-Putamen DBS models, respectively.

FIG. 8D depicts averaged LFP power of high beta band of the control models, PD (no DBS) models and PD-Putamen DBS models, respectively.

FIG. 9A depicts LFP powers of the control models, PD (no DBS) models and PD-STN DBS models, respectively.

FIG. 9B shows spectrogram for the control models, PD (no DBS) models and PD-STN DBS models, respectively.

FIG. 9C shows averaged LFP power of low beta band of the control models, PD (no DBS) models and PD-Putamen DBS models, respectively.

FIG. 9D shows averaged LFP power of high beta band of the control models, PD (no DBS) models and PD-Putamen DBS models, respectively.

FIG. 10A shows LFP powers of the control models, PD (no DBS) models and PD-GPi DBS models, respectively.

FIG. 10B shows spectrogram for the control models, PD (no DBS) models and PD-GPi DBS models, respectively.

FIG. 10C shows averaged LFP power of low beta band of the control models, PD (no DBS) models and PD-GPi DBS models, respectively.

FIG. 10D shows averaged LFP power of high beta band of the control models, PD (no DBS) models and PD-GPi DBS models, respectively.

FIG. 11 depicts an experiment paradigm for the evaluating effect of DBS on upstream (putamen) and downstream (STN/GPi) pathway of motor control in rotenone induced PD mice models.

FIG. 12A depicts LFP powers of the control models, PD (no DBS) models and PD-Putamen DBS models, respectively.

FIG. 12B depicts spectrogram for the control models, PD (no DBS) models and PD-Putamen DBS models, respectively.

FIG. 12C depicts averaged LFP power of low beta band of the control models, PD (no DBS) models and PD-Putamen DBS models, respectively.

FIG. 12D depicts averaged LFP power of high beta band of the control models, PD (no DBS) models and PD-Putamen DBS models, respectively.

FIG. 13A depicts LFP powers of the control models, PD (no DBS) models and PD-STN DBS models, respectively.

FIG. 13B depicts spectrogram for the control models, PD (no DBS) models and PD-STN DBS models, respectively.

FIG. 13C depicts averaged LFP power of low beta band of the control models, PD (no DBS) models and PD-Putamen DBS models, respectively.

FIG. 13D depicts averaged LFP power of high beta band of the control models, PD (no DBS) models and PD-Putamen DBS models, respectively.

FIG. 14A depicts LFP powers of the control models, PD (no DBS) models and PD-GPi DBS models, respectively.

FIG. 14B depicts spectrogram for the control models, PD (no DBS) models and PD-GPi DBS models, respectively.

FIG. 14C depicts averaged LFP power of low beta band of the control models, PD (no DBS) models and PD-GPi DBS models, respectively.

FIG. 14D depicts averaged LFP power of high beta band of the control models, PD (no DBS) models and PD-GPi DBS models, respectively.

FIG. 15 depicts an experiment paradigm for comparing effects of closed-loop and open-loop DBS on improvement of the motor symptoms of rotenone induced PD models.

FIG. 16 depicts the total number of stimulations applied in the closed-loop and open-loop DBS to rotenone induced PD models, respectively.

FIGS. 17A-17C depict the open field test results, narrow beam walk test results and pole climb test results for the control, rotenone induced PD (no DBS), PD+open-loop DBS and PD+closed-loop DBS models, respectively.

FIGS. 18A-18C depict firing rate, number of bursts and interspike interval in the putamen of the control, rotenone induced PD (no DBS), PD+open-loop DBS and PD+closed-loop DBS models, respectively.

FIG. 19 depicts an experiment paradigm for comparing effects of closed-loop and open-loop DBS on improvement of the motor symptoms of 6-OHDA induced PD models.

FIG. 20 depicts the total number of stimulations applied in the closed-loop and open-loop DBS to 6-OHDA induced PD models, respectively.

FIGS. 21A-21C depict the open field test results, narrow beam walk test results and pole climb test results for the control, 6-OHDA induced PD (no DBS), PD+open-loop DBS and PD+closed-loop DBS models, respectively.

FIGS. 22A-22C depict firing rate, number of bursts and interspike interval in the putamen of the control, 6-OHDA induced PD (no DBS), PD+open-loop DBS and PD+closed-loop DBS models, respectively.

### DETAILED DESCRIPTION

In the following description, embodiments of the present invention are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In accordance with a first aspect of the present invention, a neurostimulation system is provided to apply deep brain stimulation (DBS) technology to regulate the abnormal motor movement of a subject. In particular, the neurostimulation system herein is capable of stimulating deep brain nuclei of the subject for regulating Parkinson's disease-associated motor symptoms, such as tremor, slowed movement and impaired posture and balance, and thereby alleviating the symptoms. It would be appreciated that the regulation of the motor symptoms may be resulted from changes of the neuronal activities such as, but not limited to, an increase in interspike interval and a decrease in neuronal firing after deep brain stimulation. It would also be appreciated that the provided neurostimulation system can be modified for regulating other neurological disorders.

The term “subject” of the present invention in particular refers to an animal or human, in particular a mammal and most preferably a human being. The subject who is particularly benefit from the present invention is an individual who has abnormal motor movement compared to ordinary healthy individuals. Suitable devices and methods can be used to determine the regulation effect of the deep brain stimulation on the subject.

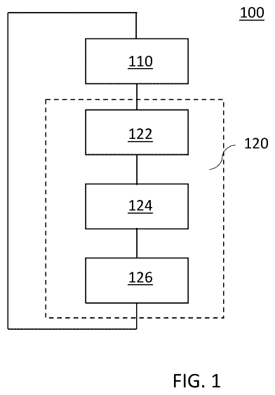

FIG. 1 depicts a schematic diagram of a closed-loop neurostimulation system 100 according to one embodiment of the present invention for regulating motor symptoms of a subject. As shown, the closed-loop neurostimulation system 100 comprises one or more neural electrodes 110 and a closed-loop stimulation generator 120 in communication with the neural electrodes 110.

The neural electrodes 110 may be implanted in a group of deep brain nuclei of the subject and configured for detecting and recording the local filed potential (LFP) signals generated in the deep brain nuclei and delivering deep brain stimulation (DBS) pulses to the deep brain nuclei. It would be appreciated that the closed-loop neurostimulation system 100 may include further electrodes such as reference electrodes and grounding electrodes during implementation, as well as connectors and batteries for establishing a complete circuit.

The stimulation at the putamen is exceptionally suitable for regulating Parkinson's disease-associated motor symptoms. The putamen is a subcortical structure of the basal ganglia and known for its role in facilitating movement.

In one embodiment, the neural electrodes may be implanted in a putamen of the subject and configured for detecting and recording local filed potential (LFP) signals generated in the putamen and delivering deep brain stimulation (DBS) pulses to the putamen.

The closed-loop stimulation generator 120 may include a data acquisition unit 122 in communication with the neural electrodes 110. The data acquisition unit 122 may be configured to: receive the LFP signals transmitted from the neural electrodes; and convert the LFP signals to LFP data. The data acquisition unit 122 may be an electronic device equipped with data acquisition software to collect LFP signals from the neural electrodes 110 and forward them to the processing unit 124 for further processing. In some embodiments, the data acquisition unit 122 may be implemented by way of a portable and compact electronic device which can be carried by or attached on the subject, depending on the configuration of the data acquisition unit 122 and the arrangement of the neurostimulation system 100. The data acquisition unit 122 may be connected to the neural electrodes 110 via a wired or wireless network. In some embodiments, the data acquisition unit 122 may further comprise local memory for storing collected LFP data.

The closed-loop stimulation generator 120 may further include a processing unit 124 in communication with the data acquisition unit 122. The processing unit 124 may be configured to: receive the LFP data from the data acquisition unit 122; analyze the LFP data to determine presence of abnormal beta band oscillation; and generate a DBS signal upon determining presence of abnormal beta band oscillation.

In particular, the processing unit 124 may determine presence of abnormal beta band oscillation by: calculating an averaged LFP power based on the received LFP data; comparing the averaged LFP power against a reference LFP power; and determining the that the subject has abnormal beta band oscillation if the averaged beta band LPF power is higher than the reference LFP power. The reference LFP power is preferably derived based on healthy individuals who have the same age and sex as the subject.

In some embodiments, the averaged LFP power may be calculated from a full range of beta band (from 13-40 Hz) LFP power spectrum. In some embodiments, the averaged LFP power may be calculated from low beta band (from 13-20 Hz) LFP power spectrum. In some embodiments, the averaged LFP power may be calculated from high beta band (from 20-40 Hz) LFP power spectrum.

Accordingly, the neurostimulation system 100 can monitor the neural activity of the subject and generate a DBS stimulation signal to the subject only when an abnormal beta band power spectrum is detected. Therefore, the provided closed-loop DBS system can reduce stimulation time and avoid adverse side effects of continuous stimulation while maintaining treatment efficacy.

It would be understood that the processing unit 124 may be configured in various shapes or incorporated with another device. In some embodiments, the processing unit 124 may be implemented by way of a portable and compact electronic device which can be carried by or attached on the subject, depending on the configuration of the processing unit 124 and the arrangement of the neurostimulation system 100. The processing unit 124 may be connected to the data acquisition unit 122 via a wired or wireless network.

In one embodiment, the processing unit 124 may be implemented with a programmable logic unit implemented with a field-programmable gate array (FPGA) or an application-specific integrated circuit (ASIC). The programmable logic unit is capable of processing real-time data at a high computational speed. For example, the programmable logic unit may be capable of completing one real world activity within one millisecond, that is have a processing data rate of 1000 data/second.

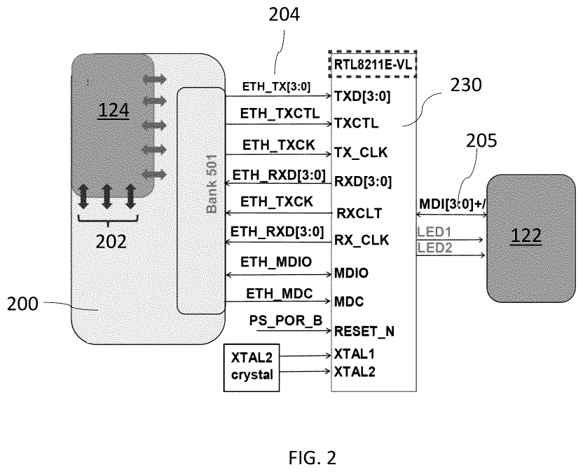

FIG. 2 depicts a schematic diagram of a FPGA board 200 with web function used for implementing the processing unit 124. As shown, the processing unit 124 may be connected to an interfacing 202 to control external devices through a driver (not shown). It may have flexible connectivity on detector front-end electronics as well as to conventional LINUX based PC for higher level processing.

An ethernet transceiver 230 may be used for data transmission between FPGA and physical layers through the RGMII bus 204. In other words, the transceiver 230 provides all the necessary physical layer functions to transmit and receive Ethernet packets over a cable 205 (e.g., CAT 5 unshielded twisted pair (UTP) cable) which are used to connect the data acquisition system 122 with a bridge server (not shown) to the FPGA board 200.

The closed-loop stimulation generator 120 may further include a pulse generation unit 126 in communication with the data processing unit and the neural electrodes. The pulse generation unit may be configured to: receive the DBS signal from the processing unit 122; generate, in response to the DBS signal, the DBS pulses to the deep brain nuclei through the neural electrodes 110.

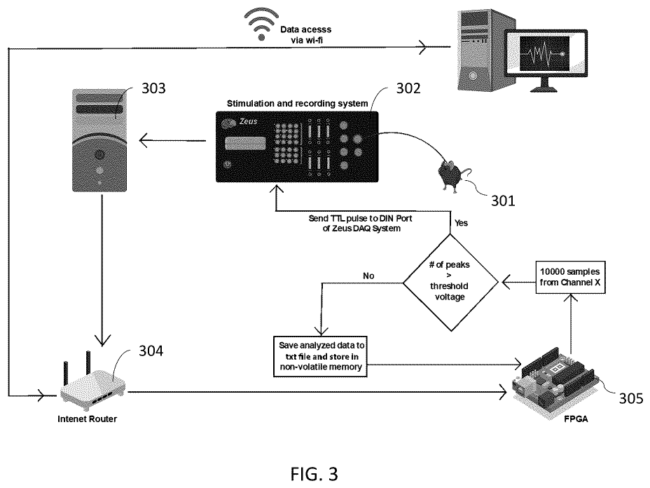

Referring to FIG. 3, in one embodiment, rotenone or 6-OHDA induced PD mouse model 301 were implanted with a 16-channel multi-electrode array (not shown). LFP signal were recorded using a data acquisition unit 302 (e.g., the Zeus DAQ system from Bio-Signal Technologies, USA) at a sampling rate of 1000 Hz. The collected data was transferred to a local computer 303, which was connected to a local bridge server 304. The bridge server 304 functions as the data bus between the local computer 303 and the FPGA board 305. Using the bridge server 304 and TCP/IP protocol, data acquisition command was sent from machine connected to the network, initiating data collecting from the mouse 301 by the Zeus System 302 to the FPGA 305. Rapid storage of the incoming UDP delivered data was managed by a temporary Redis database used for queuing of the LFP datapoints, allowing analysis and data storage to occur in parallel. Redis database is a non-relational database that utilize the volatile memory components of a system, the RAM. It allows rapid turnover of data with little latency.

In some embodiments, the closed-loop DBS may be applied using a pulse stream having 130 Hz frequency, 100 μA current amplitude and 80 μs pulse width. A pre-set program may be installed in the processing unit (i.e., FPGA board) to trigger the recording and stimulating data acquisition system to initiate the DBS in response to detection of abnormal beta band oscillation. Python program displayed on the monitor to observe the real-time running of the program. Program analyzed the data for 10 s (0-10, 20-30, 40-50 s) and delivered the DBS for 10 s (10-20, 30-40, 50-60 s) in one minute, if the beta band amplitude is above the threshold (i.e. the above-said reference LFP power). Ideally, closed-loop DBS will be delivered 3 times per minute and total number stimulation per hour will be 180. The closed-loop DBS may be applied to the subject for 1 hour on a daily basis for 7 days.

A pre-treatment recording of all 16 channels for a duration of 10 minutes was conducted for channel selection that provides the best signal to noise ratio. The data analysis code utilizes 10000 data sample per one cycle of analysis. Via the envelop method with a threshold of 200 μV, the 10 seconds of data was processed to determine if DBS pulse of 10 seconds is required. The analysis packet was then stored on the non-volatile memory component of the FPGA in txt format. Once analysis of the current 10000 samples is completed, the next 10 seconds of data is collected, and then next analysis cycle initiates and continued for one hours.

In some embodiments, the LFP data may be packaged using user datagram protocol (UDP) for data transmission between the data acquisition unit (i.e. the Zeus DAQ system) and the processing unit (i.e. FPGA). The packaged data may be sent to the FPGA via the UDP with each packet size at 1000 samples, for example, and unpackaged by the FPGA to facilitate real time analysis of the LFP data.

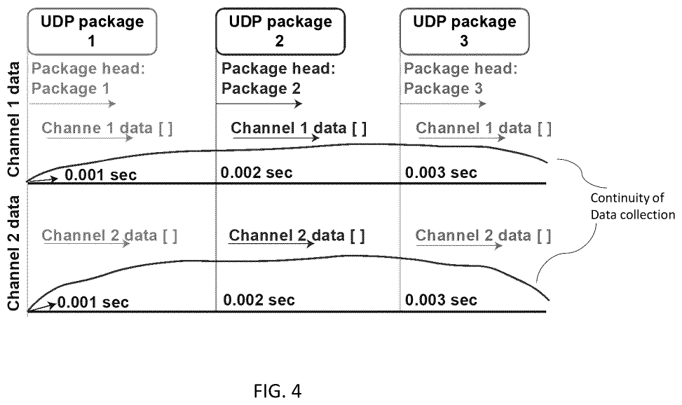

As shown in FIG. 4, two UDP communication channels 1 and 2 may be set up for data transmission. Red line denotes the continuity of data collection. Each channel may be configured to transmit three UDP data packages 1, 2 and 3 sequentially at a data transmission rate of 1000 UDP data-packages/sec. In other words, the first, second and third UPD packages (packages 1, 2 and 3) will have all the data of the channel at 0.001, 0.002 and 0.003 seconds respectively. As such, the data was sent to the processing unit (i.e. the FPGA) via the UDP with each packet size at 1000 samples. The UDP data packet is unpackaged by the FPGA to facilitate real time analysis of the LFP data. Rapid storage of the incoming UDP delivered data was managed by a temporary Redis database used for queuing of the LFP datapoints, allowing analysis and data storage to occur in parallel. Redis database is a non-relational database that utilize the volatile memory components of a system, the RAM. It allows rapid turnover of data with little latency.

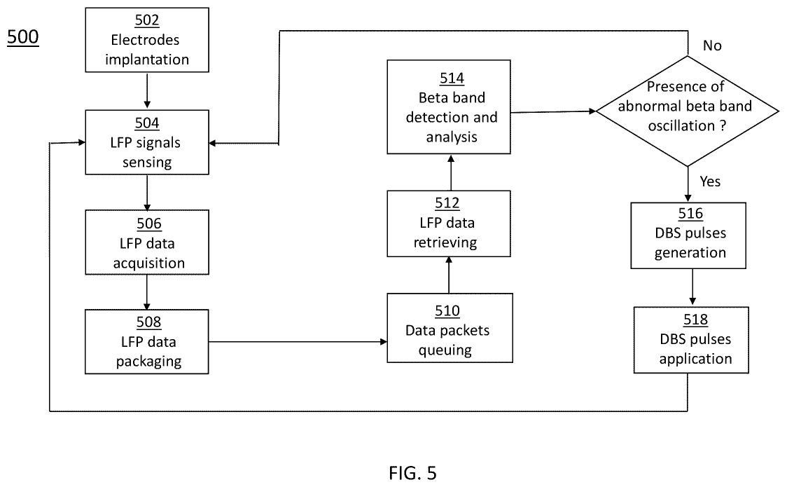

In another aspect, the present invention pertains to a method of using the closed-loop neurostimulation 100 to regulate motor symptoms of a subject. FIG. 5 shows a flowchart of a method 500 according to one embodiment of the present invention. As shown, the method 500 may comprise the following steps:

S502: implanting the neural electrodes in a group of deep brain nuclei of the subject;

S504: sensing, by the neural electrodes, LFP signals generated in the deep brain nuclei of the subject;

S506: acquiring and converting, by the data acquisition unit, the detected LFP signals to LFP data;

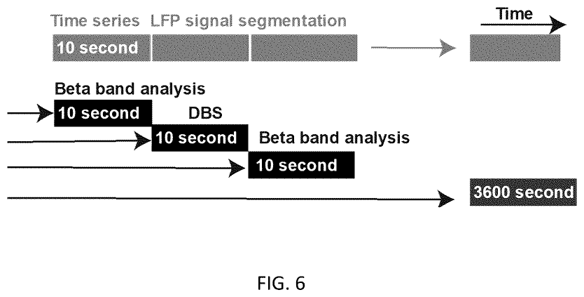

S508: packaging, by the data acquisition unit, the LFP data into data packets; in some embodiments, as shown in FIG. 6, the data packets may be generated in real-time by using sliding window method to improve the accuracy.

S510: queueing the data packets in a memory which may be a local storage in the data acquisition unit or a remote data-store;

S512: receiving, by the processing unit, the queued data packets and unpacking the data packets to retrieve the LFP data;

S514: analyzing, by the processing unit, the retrieved LFP data to determine presence of abnormal beta band power spectrum.

In some embodiments, the steps S504 to S514 may form an analysis cycle and be performed repeatedly to have a continuous detection of presence of abnormal beta band power spectrum. Upon presence of abnormal beta band oscillation is detected, the method may further comprise:

S516: commanding, by the processing unit, the pulse generation unit to generate DBS pulses to the neural electrodes; and

S518: applying, by the neural electrodes, DBS pulses to the deep brain nuclei of the subject.

### EXAMPLES

### Evaluation on 6-OHDA Induced PD Models

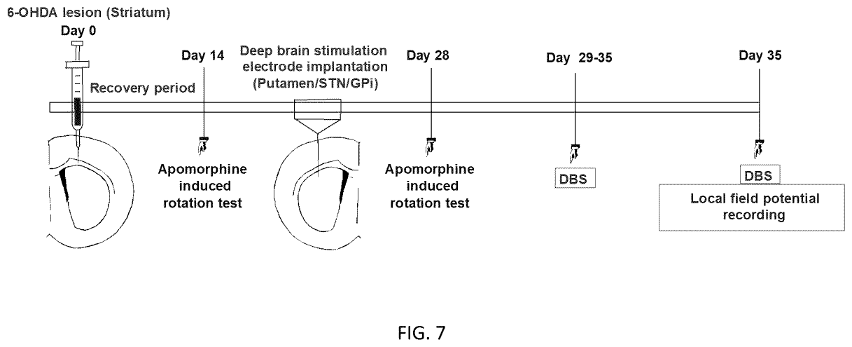

Referring to FIG. 7, the effect of DBS has been evaluated on upstream (putamen) and downstream (STN/GPi) pathway of motor control in PD mice models which were developed as PD models by injecting 6-OHDA in their striatum. Symptom was evaluated by apomorphine induced rotational behavior test. The neural electrodes were implanted in putamen/STN/GPi of three groups of PD models respectively. The PD models were then allowed to recover for 2 weeks. Each group received stimulation at 130 Hz frequency, 100 μA current amplitude, 80 μs pulse width for 1 hour daily for 7 days. LFP signals was recorded from putamen/STN/GPi in immobile awake models on day 7, immediately after DBS.

In the following description, mice with no PD are denoted as control models; PD models which have not been regulated with DBS is denoted as PD (no DBS) models; PD models which have been regulated with DBS at its putamen are denoted as PD-Putamen DBS models; PD models which have been regulated with DBS at its STN are denoted as PD-STN DBS models; and PD models which have been regulated with DBS at its GPi are denoted as PD-GPi DBS models.

LFP data was analyzed for beta band power spectrum which has a typical frequency range from 0 to 50 Hz. Total power in whole frequency spectrum was calculated to observe the full spectral change. Spectrogram was generated to check the changes in the frequency band along the time. Next, beta band power was averaged to divide into low beta (13-20 Hz) and high beta (20-40 Hz) to evaluate the efficacy of DBS.

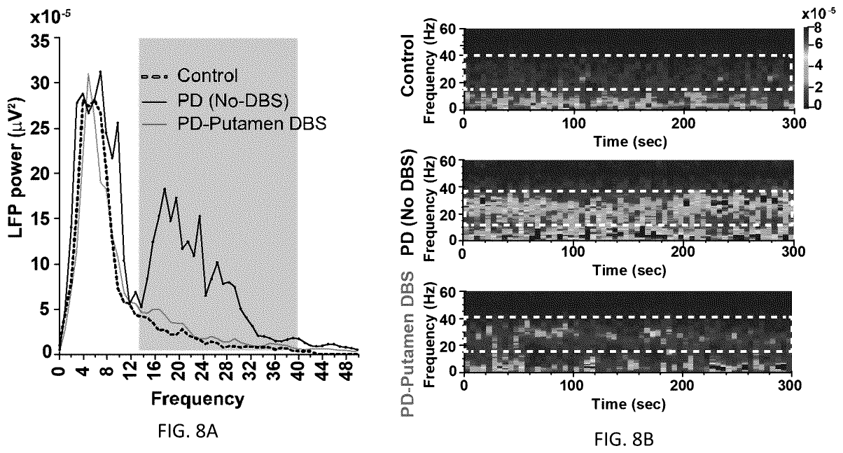

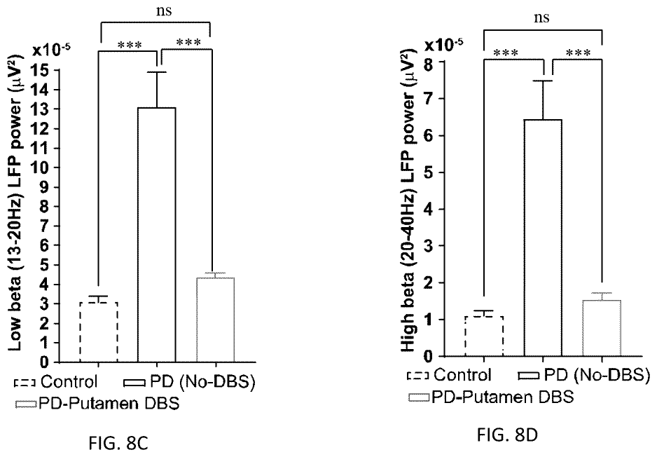

FIG. 8A shows LFP powers of the control models, PD (no DBS) models and PD-Putamen DBS models, respectively; FIG. 8B shows spectrogram for the control models, PD (no DBS) models and PD-Putamen DBS models, respectively; FIG. 8C shows averaged LFP power of low beta band of the control models, PD (no DBS) models and PD-Putamen DBS models, respectively; and FIG. 8D shows averaged LFP power of high beta band of the control models, PD (no DBS) models and PD-Putamen DBS models, respectively.

Referring to FIGS. 8A-8D, PD (no DBS) models showed significantly increased in total LFP power in low beta (13-20 Hz) and high beta band (20-40 Hz) as compared to control models (grey box), suggesting the abnormal beta band oscillation in the putamen neuron (FIG. 8A). Spectrogram of PD mice clearly showed oscillation in the beta band (white dotted line), while the control models did not show any significant change in the beta and most oscillations were restricted to low frequency (below 13 Hz) (FIG. 8B). Averaged LFP power of low beta (FIG. 8C) and high beta (FIG. 8D) was significantly increased in PD (no DBS) models as compared to control models. Overall, beta oscillation of putamen LFP in PD models clearly demonstrate its role in the pathogenesis of PD symptoms. Further, putamen DBS completely restored the beta oscillation of PD-Putamen DBS models being completely restored to normal level by significantly decreasing total LFP power (FIG. 8A), power spectrum at 13-40 Hz (FIG. 8B), average LFP power of low (FIG. 8C) and high (FIG. 8D) beta band.

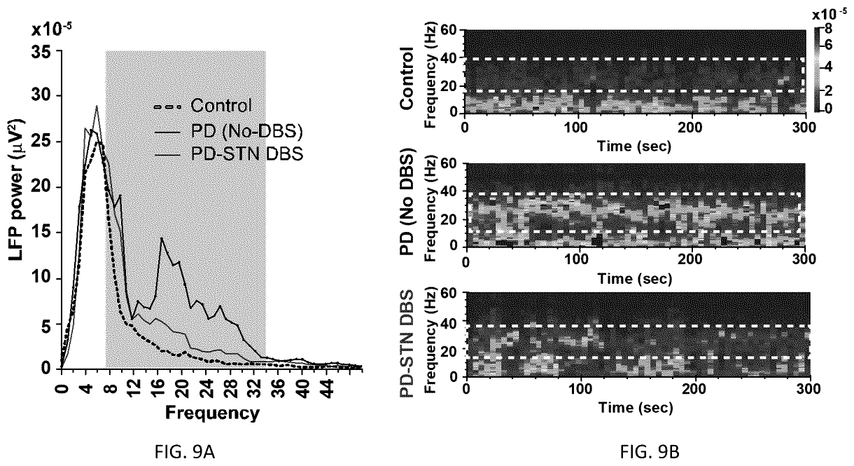

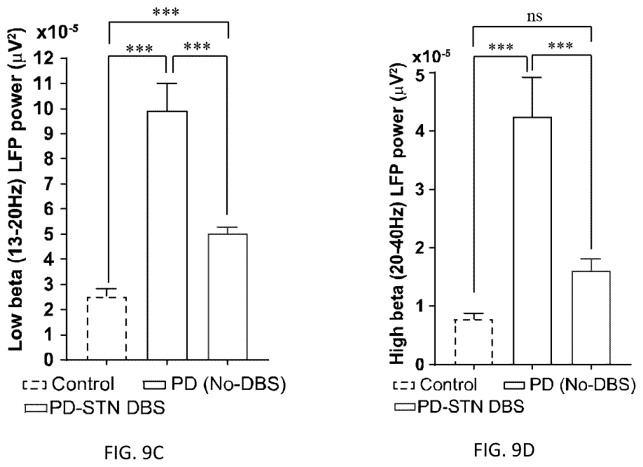

FIG. 9A shows LFP powers of the control models, PD (no DBS) models and PD-STN DBS models, respectively; FIG. 9B shows spectrogram for the control models, PD (no DBS) models and PD-STN DBS models, respectively; FIG. 9C shows averaged LFP power of low beta band of the control models, PD (no DBS) models and PD-Putamen DBS models, respectively; and FIG. 9D shows averaged LFP power of high beta band of the control models, PD (no DBS) models and PD-Putamen DBS models, respectively.

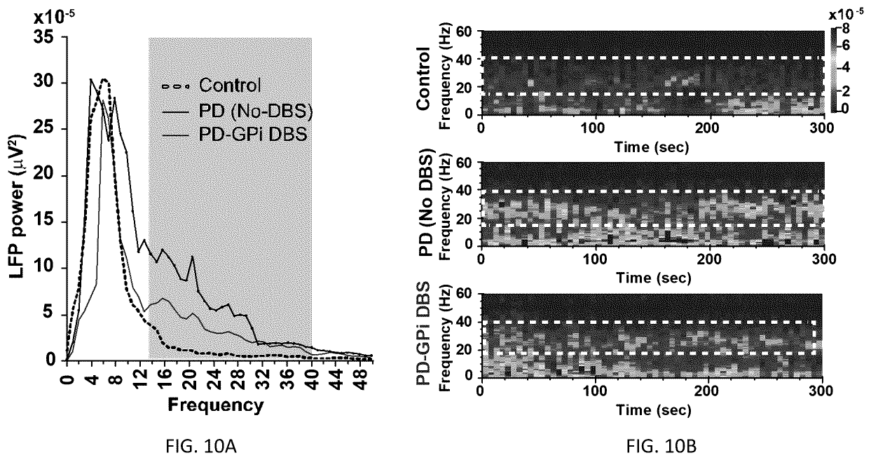

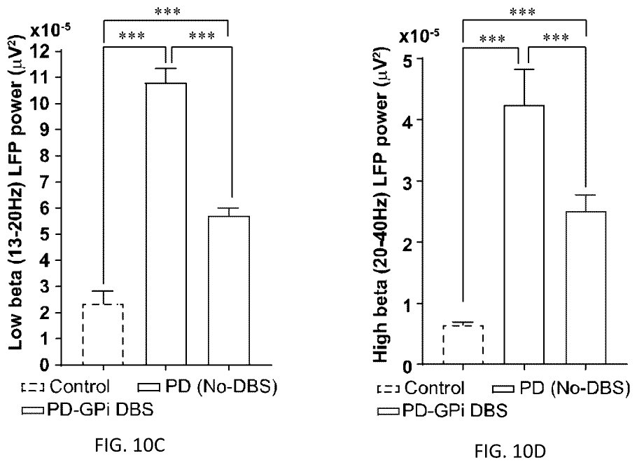

FIG. 10A shows LFP powers of the control models, PD (no DBS) models and PD-GPi DBS models, respectively; FIG. 10B shows spectrogram for the control models, PD (no DBS) models and PD-GPi DBS models, respectively; FIG. 10C shows averaged LFP power of low beta band of the control models, PD (no DBS) models and PD-GPi DBS models, respectively; and FIG. 10D shows averaged LFP power of high beta band of the control models, PD (no DBS) models and PD-GPi DBS models, respectively.

LFP power spectrum at 13-40 Hz, averaged LFP power of low and high beta band of PD-STN DBS models (FIGS. 9A-9D) and PD-GPi DBS models (FIGS. 10A-10D) are compared with control models and PD (no DBS) models. As shown, DBS models demonstrated significantly decreased beta oscillation as compared to PD (no DBS), but their beta oscillation was significantly higher as compared to control. Only PD-STN DBS mice showed their beta band oscillation power being normalized to level of the control mice. Overall, PD-putamen DBS models showed higher efficacy in normalization of beta band oscillation as compared with PD-STN DBS and GPi DBS models.

### Evaluation on Rotenone Induced PD Models

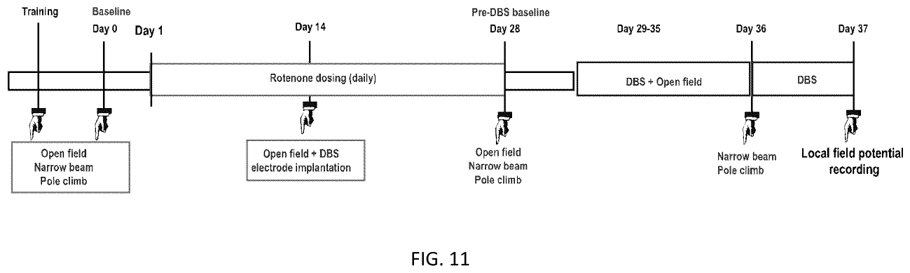

Referring to FIG. 11, the effect of DBS on rotenone induced PD mice models which were developed by daily oral administration of rotenone for 28 days are evaluated. Abnormal motor movement was assessed by open field, narrow beam walk, and pole climb test. Neural electrodes were implanted in putamen/STN/GPi of PD models showing 50% decrease in total distance travelled of open filed test. After recovery and pre-DBS baseline, each group were subjected to receive DBS at 130 Hz frequency, 100 μA current amplitude, 80 μs pulse width for 1-hour daily basis for 7 days. LFP was recorded in immobile wake mouse immediately after DBS.

In the following description, mice with no PD are denoted as control models; PD models which have not been regulated with DBS is denoted as PD (no DBS) models; PD models which have been regulated with DBS at its putamen are denoted as PD-Putamen DBS models; PD models which have been regulated with DBS at its STN are denoted as PD-STN DBS models; and PD models which have been regulated with DBS at its GPi are denoted as PD-GPi DBS models.

Similarly, LFP data was analyzed for beta band oscillation which has a typical frequency range from 0 to 50 Hz. Total power in whole frequency spectrum was calculated to observe the full spectral change. Spectrogram was generated to check the changes in the frequency band along the time. Next, beta band power was averaged to divide into low beta (13-20 Hz) and high beta (20-40 Hz) to evaluate the efficacy of DBS.

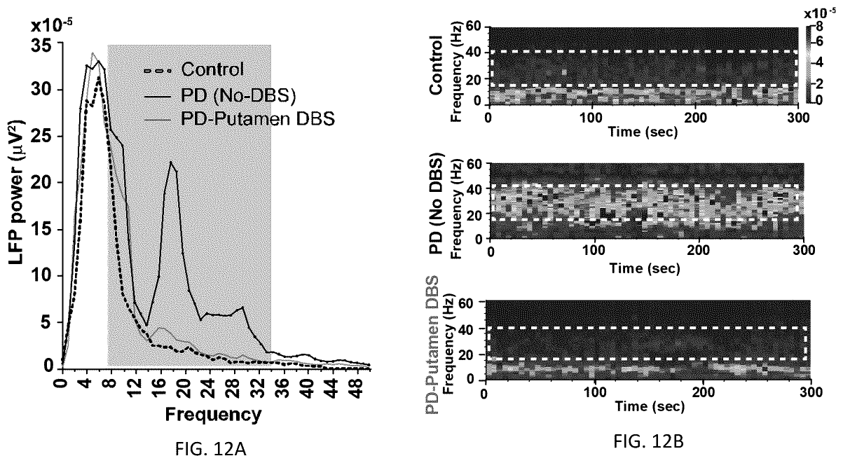

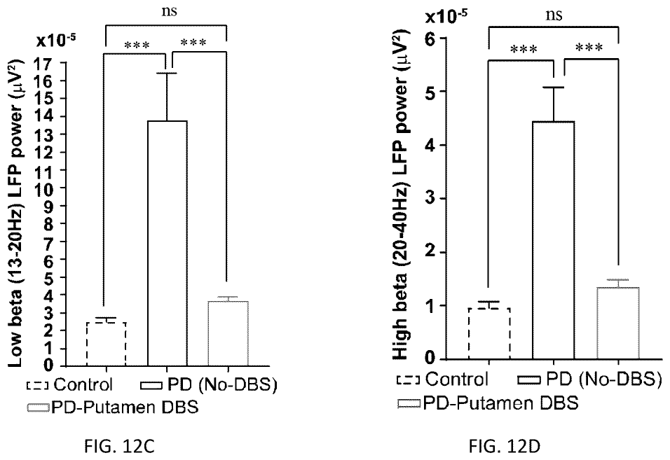

FIG. 12A shows LFP powers of the control models, PD (no DBS) models and PD-Putamen DBS models, respectively; FIG. 12B shows spectrogram for the control models, PD (no DBS) models and PD-Putamen DBS models, respectively; FIG. 12C shows averaged LFP power of low beta band of the control models, PD (no DBS) models and PD-Putamen DBS models, respectively; and FIG. 12D shows averaged LFP power of high beta band of the control models, PD (no DBS) models and PD-Putamen DBS models, respectively.

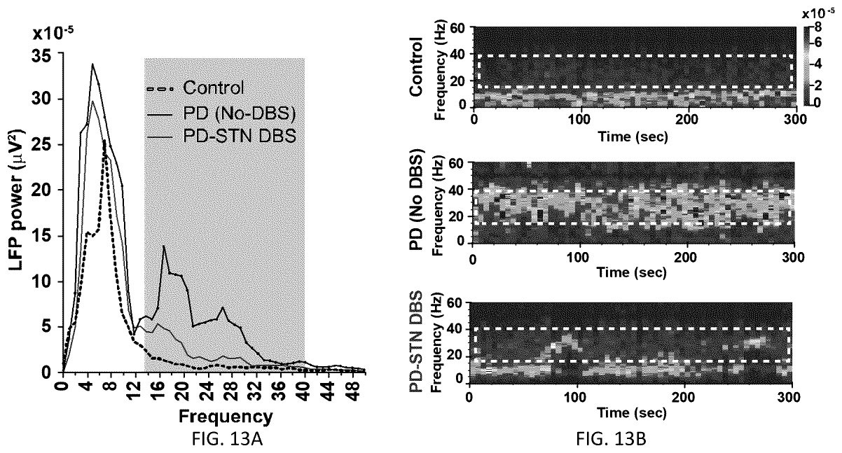

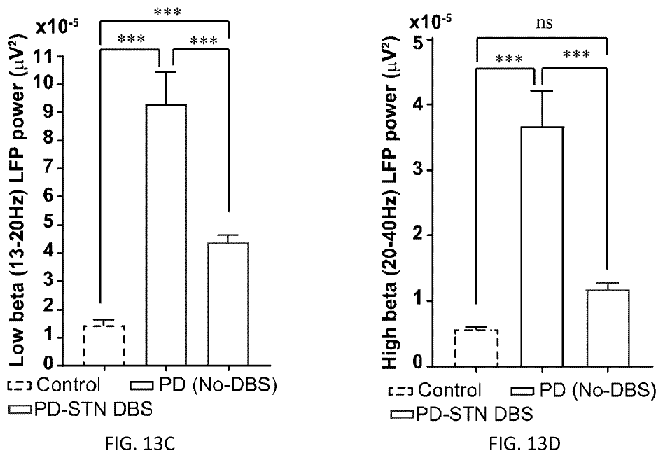

FIG. 13A shows LFP powers of the control models, PD (no DBS) models and PD-STN DBS models, respectively; FIG. 13B shows spectrogram for the control models, PD (no DBS) models and PD-STN DBS models, respectively; FIG. 13C shows averaged LFP power of low beta band of the control models, PD (no DBS) models and PD-Putamen DBS models, respectively; and FIG. 13D shows averaged LFP power of high beta band of the control models, PD (no DBS) models and PD-Putamen DBS models, respectively.

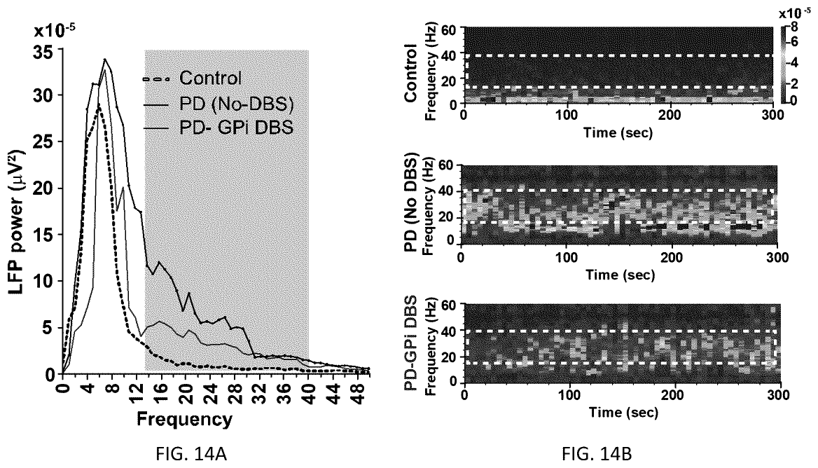

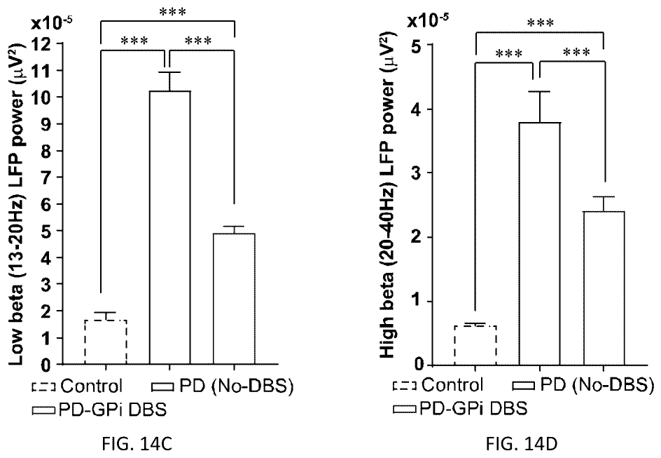

FIG. 14A shows LFP powers of the control models, PD (no DBS) models and PD-GPi DBS models, respectively; FIG. 14B shows spectrogram for the control models, PD (no DBS) models and PD-GPi DBS models, respectively; FIG. 14C shows averaged LFP power of low beta band of the control models, PD (no DBS) models and PD-GPi DBS models, respectively; and FIG. 14D shows averaged LFP power of high beta band of the control models, PD (no DBS) models and PD-GPi DBS models, respectively.

Referring to FIGS. 12A-12D, 13A-13D, 14A-14D, PD models showed significantly increased total LFP power, power spectrum (13-40 Hz), average LFP power of low and high beta in putamen (FIGS. 12A-12D), STN (FIGS. 13A-13D) and GPi (FIGS. 14A-14D) as compared to control models, while putamen/STN/GPi DBS significantly showed decreased beta oscillation power. Low and high beta band oscillation power was restored to normal in putamen DBS group (FIGS. 12A-12D), while STN-DBS showed restoration in high beta band only (FIGS. 13A-13D). However, low and high beta band power was significantly higher in GPi DBS as compared to control models (FIGS. 14A-14D).

Overall, the rotenone induced PD mice models showed abnormal beta oscillations in the putamen, STN and GPi suggesting its role in pathogenesis of PD. PD-putamen DBS models have their beta oscillations completely restored to normal level, while PD-STN DBS and GPi DBS models did not.

### Comparison Between Closed-Loop DBS and Open-Loop DBS

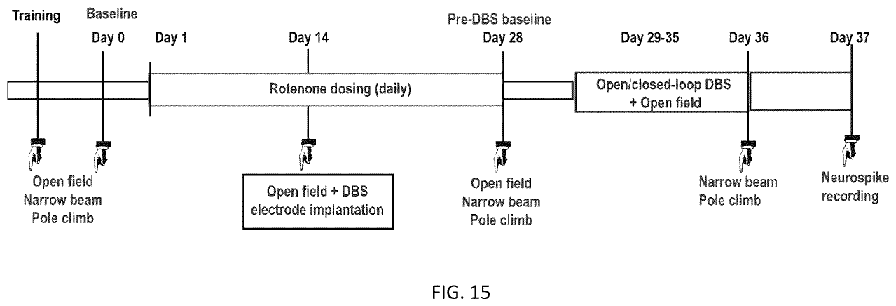

FIG. 15 shows an experiment paradigm for comparing effects of closed-loop and open-loop DBS on improvement of the motor symptoms of rotenone induced PD models. In the closed-loop DBS, the rotenone induced PD mice received DBS at 130 Hz frequency, 100 μA current amplitude, 80 μs pulse width for 1 hour on a daily basis for 7 days (day 29-35), through neural electrodes implanted in their putamen. A pre-set program was installed on a FPGA closed loop protype (acting as the processing unit) to trigger the recording and stimulating the Zeus DAQ system to initiate the DBS in response to detection of abnormal beta band oscillation. The closed-loop DBS were delivered 3 times per minute and total number stimulation per hour was 180. Similar amount of stimulation time was followed (30 minutes) in the open loop-DBS for comparison. Open field test on day 35, pole climb, and narrow beam walk on day 36 was performed during DBS to evaluate the efficacy.

In the following description, mice with no PD are denoted as control models; PD models which have not been regulated with DBS is denoted as PD (no DBS) models; PD models which have been regulated with closed-loop DBS are denoted as PD+closed-loop DBS models; and PD models which have been regulated with open-loop DBS are denoted as PD+open-loop DBS models.

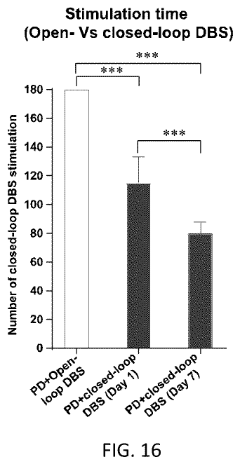

FIG. 16 shows the total number of stimulations applied in the closed-loop and open-loop DBS to rotenone induced PD models, respectively. Referring to FIG. 16, the total number of stimulations in closed-loop DBS on day 7 was decreased by 55.4% as compared with open-loop DBS.

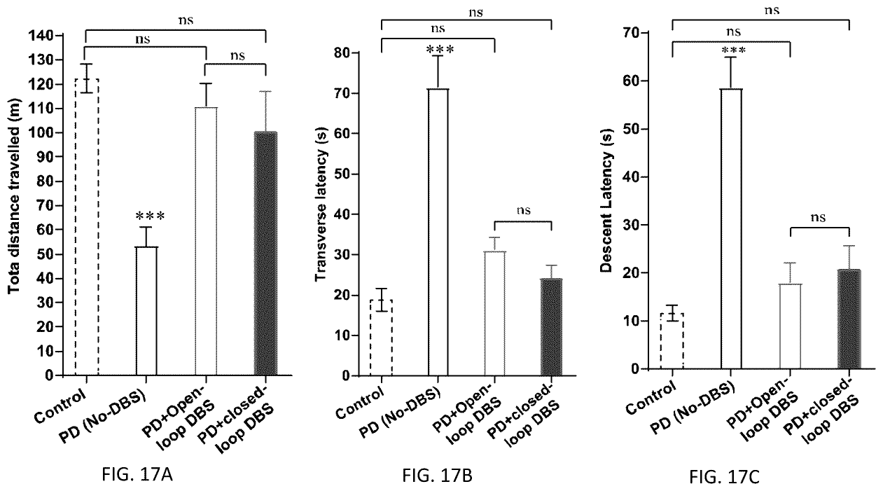

FIGS. 17A-17C show the open field test results, narrow beam walk test results and pole climb test results for the control, rotenone induced PD (no DBS), PD+open-loop DBS and PD+closed-loop DBS models, respectively. As shown, the PD+closed-loop DBS models restored total distance travelled in open field test (FIG. 17A), transverse latency in narrow beam walk test (FIG. 17B) and descent latency in pole climbing test (FIG. 17C) to normal level of the control mice. There was no significant difference between open-and closed-loop DBS. Motor activity was completely restored, as the putamen closed-loop DBS treated group did not show significant difference as compared to control mice.

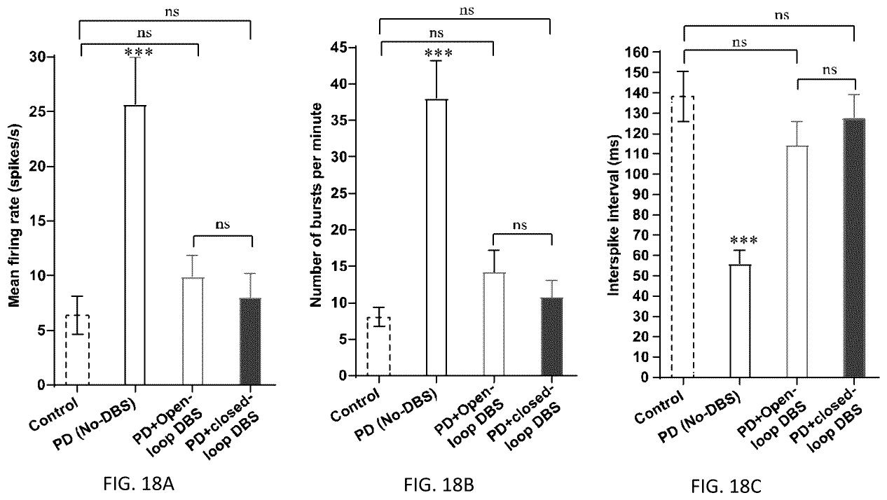

FIGS. 18A-18C show firing rate, number of bursts and interspike interval in the putamen of the control, rotenone induced PD (no DBS), PD+open-loop DBS and PD+closed-loop DBS models, respectively. Similar trend was observed in electrophysiology of putamen neurons after closed-loop DBS. Closed-loop DBS restored (or regulated) the neuronal firing rate (FIG. 18A), number of bursts (FIG. 18B), and interspike interval (FIG. 18C) to normal level of control mice and there was no significant difference as compared to open-loop DBS.

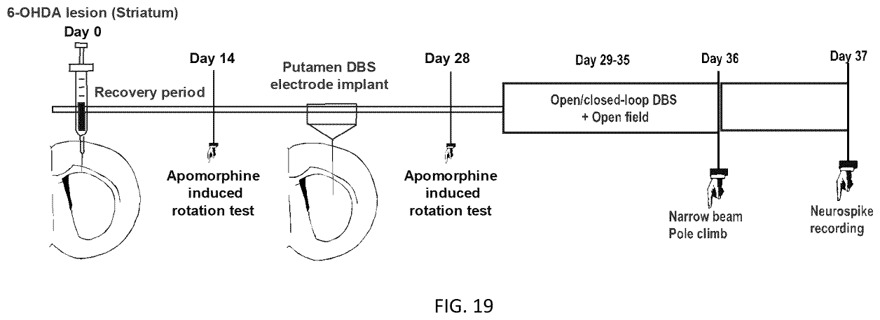

FIG. 19 shows an experiment paradigm for comparing effects of closed-loop and open-loop DBS on improvement of the motor symptoms of 6-OHDA induced PD models. Similarly, in the closed-loop DBS, the 6-OHDA induced PD mice received DBS at 130 Hz frequency, 100 μA current amplitude, 80 μs pulse width for 1 hour on a daily basis for 7 days (day 29-35), through neural electrodes implanted in their putamen. A pre-set program was installed on a FPGA closed loop protype (acting as the processing unit) to trigger the recording and stimulating the Zeus DAQ system to initiate the DBS in response to detection of abnormal beta band oscillation. The closed-loop DBS were delivered 3 times per minute and total number stimulation per hour was 180. Similar amount of stimulation time was followed (30 minutes) in the open loop-DBS for comparison. Open field test on day 35, pole climb, and narrow beam walk on day 36 was performed during DBS to evaluate the efficacy.

In the following description, mice with no PD are denoted as control models; PD models which have not been regulated with DBS is denoted as PD (no DBS) models; PD models which have been regulated with closed-loop DBS are denoted as PD+closed-loop DBS models; and PD models which have been regulated with open-loop DBS are denoted as PD+open-loop DBS models

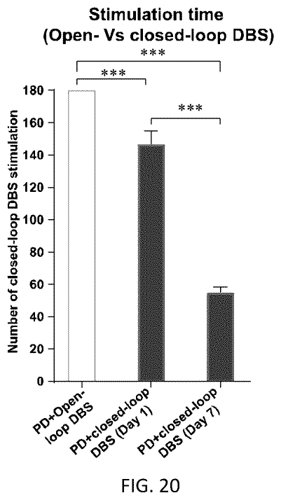

FIG. 20 shows the total number of stimulations applied in the closed-loop and open-loop DBS to 6-OHDA induced PD models, respectively. Referring to FIG. 20, the total number of stimulations in closed-loop DBS on day 7 was decreased by 69.3% as compared with open-loop DBS.

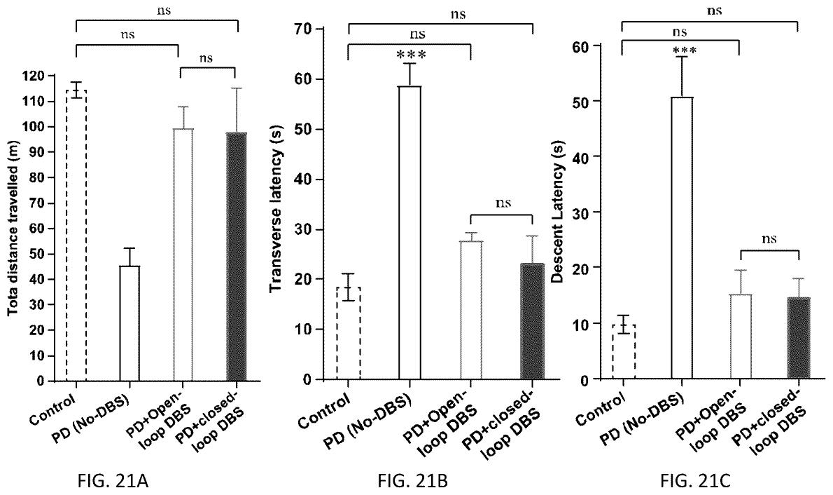

FIGS. 21A-21C show the open field test results, narrow beam walk test results and pole climb test results for the control, 6-OHDA induced PD (no DBS), PD+open-loop DBS and PD+closed-loop DBS models, respectively. As shown, PD symptoms were restored (or regulated) to normal after 7 days closed-loop DBS, where total distance travelled in open field test (FIG. 21A), transverse latency in narrow beam walk test (FIG. 21B) and descent latency in pole climbing test (FIG. 21C) did not showed significant difference as compared to open-loop DBS.

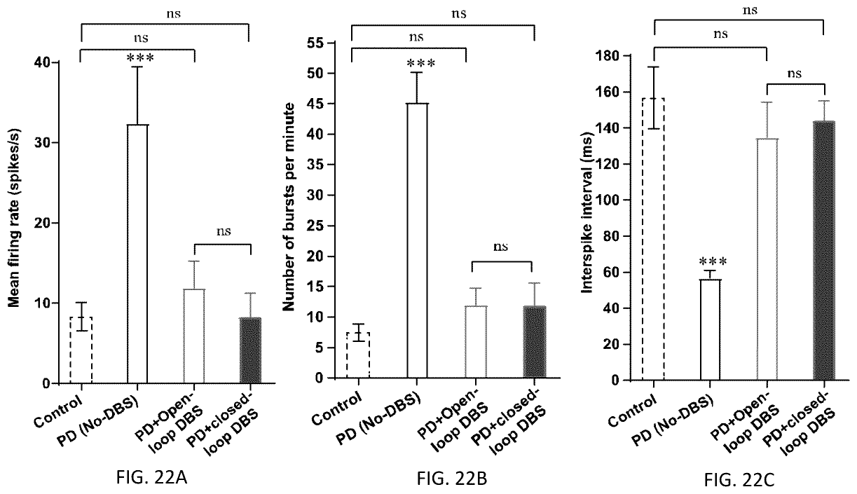

FIGS. 22A-22C show firing rate, number of bursts and interspike interval in the putamen of the control, 6-OHDA induced PD (no DBS), PD+open-loop DBS and PD+closed-loop DBS models, respectively. Electrophysiology of putamen neurons were normalized to control level after closed-loop DBS. Neuronal firing rate (FIG. 22A), number of bursts (FIG. 22B), and interspike interval (FIG. 22C) did not show significant difference between open and closed-loop DBS.

Overall, the FPGA-based closed-loop DBS prototype showed restoration of motor symptoms to normal level as evaluated by open filed, narrow beam, pole climb tests in rotenone-induced as well as 6-OHDA-induced PD mice models. Closed-loop DBS decreased the total stimulation by 69.3% in 6-OHDA PD model and 55.4% in rotenone PD model

The embodiments were chosen and described in order to best explain the principles of the invention and its practical application, thereby enabling others skilled in the art to understand the invention for various embodiments and with various modifications that are suited to the particular use contemplated. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations. While the apparatuses disclosed herein have been described with reference to particular structures, shapes, materials, composition of matter and relationships . . . etc., these descriptions and illustrations are not limiting. Modifications may be made to adapt a particular situation to the objective, spirit and scope of the present disclosure. All such modifications are intended to be within the scope of the claims appended hereto.

1. A closed-loop neurostimulation system for regulating motor symptoms of a subject, comprising:
one or more neural electrodes for being implanted in a group of deep brain nuclei of the subject to record local filed potential (LFP) signals generated in the deep brain nuclei and deliver deep brain stimulation (DBS) pulses to the deep brain nuclei; and
a closed-loop stimulation generator in communication with the neural electrodes and including:
a data acquisition unit in communication with the neural electrodes and configured to: receive the LFP signals transmitted from the neural electrodes; and convert the LFP signals to LFP data;
a processing unit in communication with the data acquisition unit and configured to: receive the LFP data from the data acquisition unit; analyze the LFP data to determine presence of abnormal beta band oscillation; and generate a DBS signal upon determining presence of abnormal beta band oscillation; and
a pulse generation unit in communication with the data processing unit and the neural electrodes and configured to: receive the DBS signal from the processing unit; and generate, in response to the DBS signal, the DBS pulses to the deep brain nuclei through the neural electrodes.

2. The closed-loop neurostimulation system of claim 1, wherein the processing unit is implemented with a field-programmable gate array.

3. The closed-loop neurostimulation system of claim 1, wherein the processing unit is further configured to:
calculate an averaged LFP power based on the received LFP data;
compare the averaged LFP power against a reference LFP power; and
determine that the subject has abnormal beta band oscillation if the averaged beta band LPF power is higher than the reference LFP power.

4. The closed-loop neurostimulation system of claim 1, wherein the averaged LFP power is calculated from a beta band LFP power spectrum in a range from 13 to 40 Hz.

5. The closed-loop neurostimulation system of claim 4, wherein the averaged LFP power is calculated from a low beta band LFP power spectrum in a range from 13 to 20 Hz.

6. The closed-loop neurostimulation system of claim 4, wherein the averaged LFP power is calculated from a low beta band LFP power spectrum in a range from 20 to 40 Hz.

7. The closed-loop neurostimulation system of claim 1, wherein the neural electrodes communicate with the processing unit via a wireless network.

8. The closed-loop neurostimulation system of claim 1, wherein the neural electrodes communicate with the processing unit via a wired network.

9. The closed-loop neurostimulation system of claim 1, wherein the data acquisition unit is further configured to package the LFP data using user datagram protocol (UDP) and transmitted packaged LFP data the processing unit.

10. The closed-loop neurostimulation system of claim 1, the data acquisition unit comprises a local memory for storing and queuing up the packaged LFP data.

11. The closed-loop neurostimulation system of claim 1, wherein the group of deep brain nuclei is Putamen.

12. A closed-loop neurostimulation method for regulating motor symptoms of a subject, comprising:
implanting one or more neural electrodes in a group of deep brain nuclei of the subject;
sensing, by the neural electrodes, local filed potential (LFP) signals generated in the deep brain nuclei of the subject;
acquiring and converting, by a data acquisition unit, the detected LFP signals to LFP data;
packaging, by the data acquisition unit, the LFP data into data packets;
queueing, by the data acquisition unit, the data packets in a memory;
receiving, by a processing unit, the queued data packets from the memory;
unpacking, by the processing unit, the data packets to retrieve the LFP data;
analyzing, by the processing unit, the retrieved LFP data to determine presence of abnormal beta band oscillation;
commanding, by the processing unit, a pulse generation unit to generate DBS pulses to the neural electrodes upon presence of abnormal beta band oscillation; and
applying, by the neural electrodes, deep brain stimulation (DBS) pulses to the deep brain nuclei of the subject.

13. The closed-loop neurostimulation method according to claim 12, wherein analyzing the retrieved LFP data to determine presence of abnormal beta band oscillation comprises:
calculating an averaged LFP power based on the received LFP data;
comparing the averaged LFP power against a reference LFP power; and
determining that the subject has abnormal beta band oscillation if the averaged beta band LPF power is higher than the reference LFP power.

14. The closed-loop neurostimulation method according to claim 13, wherein the averaged LFP power is calculated from a beta band LFP power spectrum in a range from 13 to 40 Hz.

15. The closed-loop neurostimulation method according to claim 14, wherein the averaged LFP power is calculated from a low beta band LFP power spectrum in a range from 13 to 20 Hz.

16. The closed-loop neurostimulation method according to claim 14, wherein the averaged LFP power is calculated from a low beta band LFP power spectrum in a range from 20 to 40 Hz.

17. The closed-loop neurostimulation method according to claim 12, wherein the LFP data are packaged in real-time using a sliding window method.

18. The closed-loop neurostimulation method according to claim 12, wherein the memory a local storage in the data acquisition unit.

19. The closed-loop neurostimulation method according to claim 12, wherein the memory is a remote data-store.

20. The closed-loop neurostimulation method according to claim 12, wherein the group of deep brain nuclei is Putamen.
